(19) United States
(12) Patent Application Publication
(10) Pub. No.: US 2004/0215163 A1
(43) Pub. Date: Oct. 28, 2004

(54) Intravascular heat exchange catheter with multiple coolant inlet holes to balloon

(76) Inventors: Blair D. Walker, Mission Viejo, CA (US); Wayne Arthur Noda, Mission Viejo, CA (US)

Correspondence Address: ROGITZ & ASSOCIATES, 750 B STREET, SUITE 3120, SAN DIEGO, CA, 92101, US

(21) Appl. No.: 10/425,137

(22) Filed: Apr. 28, 2003

(51) Int. Cl. A61M 31/00

(52) U.S. Cl. 604/509; 604/113

(57) ABSTRACT

Plural coolant inlet holes are provided into a balloon of a closed loop intravascular cooling catheter to increase the effective surface area of the balloon available for heat exchange. The coolant may be a mixture of water and ethanol.

### I. FIELD OF THE INVENTION

The present invention relates generally to intravascular heat exchange catheters.

### II. BACKGROUND OF THE INVENTION

Intravascular catheters have been introduced for controlling patient temperature. Typically, a coolant such as saline is circulated through an intravascular heat exchange catheter, which is positioned in the patient's bloodstream, to cool or heat the blood as appropriate for the patient's condition. The coolant is warmed or cooled by a computer-controlled heat exchanger that is external to the patient and that is in fluid communication with the catheter.

For example, intravascular heat exchange catheters can be used to combat potentially harmful fever in patients suffering from neurological and cardiac conditions such as stroke, subarachnoid hemorrhage, intracerebral hemorrhage, cardiac arrest, and acute myocardial infarction, or to induce therapeutic hypothermia in such patients. Further, such catheters can be used to rewarm patients after, e.g., cardiac surgery or for other reasons. Intravascular catheters afford advantages over external methods of cooling and warming, including more precise temperature control and more convenience on the part of medical personnel.

The following U.S. patents, all of which are incorporated herein by reference, disclose various intravascular catheters/systems/methods: U.S. Pat. Nos. 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559.

The present invention has critically recognized the need to optimize the heat exchange capacities of intravascular catheters, both by ensuring better heating and/or cooling of the heat exchange surfaces of the catheters and by increasing the temperature differential between the heat exchange fluid flowing through the catheter and the patient's body temperature.

### SUMMARY OF THE INVENTION

A system for treating a patient includes a heat exchange catheter configured for placement in the patient to induce hypothermia in the patient when heat exchange fluid is circulated through the catheter. The catheter includes a catheter body, at least one enclosure-forming structure on the body, and plural heat exchange fluid inlet holes in the catheter body. The inlet holes form respective passageways for heat exchange fluid into the enclosure-forming structure. At least one outlet hole may also be provided in the body.

Preferably, the inlet holes are smaller than the outlet hole. A heat exchanger may also be provided for supplying heat exchange fluid to the catheter and for receiving heat exchange fluid from the catheter in a closed circuit. The heat exchange fluid may include a substance such as ethanol that has a freezing point below zero degrees Celsius (0° C.) and water.

In another aspect, a method for treating a patient includes providing a closed loop heat exchange catheter having at least one heat exchange element, and directing heat exchange fluid into the element from the catheter through at least two inlet holes. The method also includes removing fluid from the balloon through at least one outlet hole.

In still another aspect, a system for effecting heat exchange with a patient includes a heat exchange catheter configured for placement in the patient to induce hypothermia in the patient when heat exchange fluid is circulated through the catheter. The system also includes a heat exchanger that supplies heat exchange fluid to the catheter and that receives heat exchange fluid from the catheter in a closed circuit. The heat exchange fluid includes at least one substance having a freezing point below zero degrees Celsius (0° C.), and water.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a perspective view of the present intravascular heat exchange catheter, schematically showing a medicament source and coolant source in an exploded relationship with the catheter; and

FIG. 2 is a partial cross-sectional diagram as seen along the line 2-2 in FIG. 1.

### DETAILED DESCRIPTION OF THE PREFERRED EMBODIMENT

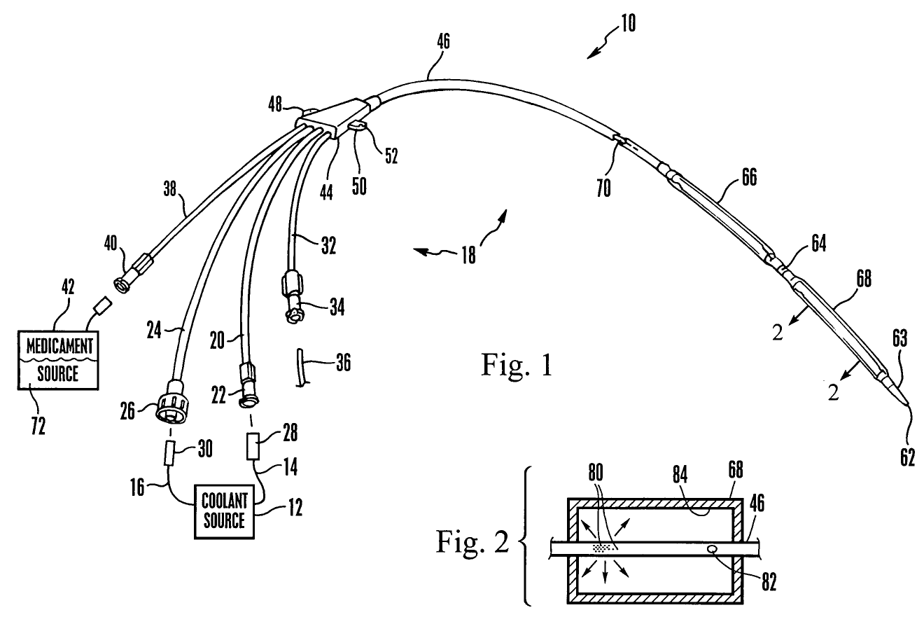

Referring initially to FIG. 1, a therapeutic catheter system, generally designated 10, is shown for establishing and maintaining hypothermia in a patient, or for attenuating a fever spike in a patient and then maintaining normal body temperature in the patient. Commencing the description of the system 10 at the proximal end, as shown the system 10 includes a heat exchange fluid source 12 that can be a water-bath heat exchange system or a TEC-based heat exchange system such as any of the systems disclosed in one or more of the above-referenced patents. In any case, the heat exchange fluid source provides warmed or cooled heat exchange fluid such as saline through, a heat exchange fluid supply line 14, and heat exchange fluid is returned to the source 12 via a heat exchange fluid return line 16.

In addition to using saline, the heat exchange fluid can be a mixture of water (or saline) and a substance that will permit the heat exchange fluid to be cooled to a temperature below zero degrees Celsius (0° C.) without solidifying and thereby achieve a higher differential temperature with the body when cooling, thus permitting more rapid patient cooling. An exemplary substance for this purpose is ethanol.

A catheter, generally designated 18, includes a source tube 20 terminating in a fitting such as a female Luer fitting 22. Also, the catheter 18 has a return tube 24 terminating in a fitting such a male Luer fitting 26. The fittings 22, 26 can be selectively engaged with complementary fittings 28, 30 of the lines 14, 16 to establish a closed circuit heat exchange fluid path between the catheter 18 and heat exchange fluid source 12. The catheter 18 may be any one of the catheters set forth in the above-referenced patents. An exemplary catheter is set forth herein for illustration purposes.

Additionally, the preferred non-limiting catheter 18 includes a guide wire and primary infusion tube 32 that terminates in a fitting such as a female Luer 34. A guide wire 36 can be advanced through the tube 32 in accordance with central venous catheter placement principles, or medicament or other fluid can be infused through the guide wire and primary infusion tube 32. Moreover, a secondary infusion tube 38 with female Luer fitting 40 can be selectively engaged with a medicament source 42 for infusing fluid from the source 42 through the secondary tube 38 in accordance with present principles discussed below.

As discussed further below, the tubes 20, 24, 32, 38 are held in a distally-tapered connector manifold 44. As also set forth further below, the connector manifold 44 establishes respective pathways for fluid communication between the tubes 20, 24, 32, 38 and respective lumens in a catheter body 46.

A suture anchor 48 advantageously can be formed on the connector manifold 44 for suturing the catheter 18 to a patient. In one intended environment, the suture anchor 48 includes opposed ears 50 formed with respective suture holes 52. Other equivalent anchor structure can be used to hold the catheter 18 onto the patient, however, including surgical tape. When the catheter is a so-called Swan-Ganz catheter, i.e., a catheter of the type disclosed in U.S. Pat. No. 3,995,623, incorporated herein by reference, the anchor 48 typically would not be provided.

The exemplary catheter body 46 may include at least two lumens, and in the preferred embodiment the catheter body 46 includes at least four lumens. More specifically, the catheter body 46 can define a heat exchange fluid supply lumen, a heat exchange fluid return lumen, a guide wire lumen, and a secondary infusion lumen. A lumen can also be provided for holding a wire or wires that are attached to one or more distally-located sensors, such as temperature sensors, pressure sensors, gas sensors, and electrical sensors.

In any case, the connector manifold 44 establishes a pathway for fluid communication between the heat exchange fluid supply tube 20 and the heat exchange fluid supply lumen of the catheter. Likewise, the connector manifold 44 establishes a pathway for fluid communication between the heat exchange fluid return tube 24 and the heat exchange fluid return lumen. Further, the connector manifold 44 establishes a pathway for fluid communication between the guide wire and primary infusion tube 32, and the guide wire lumen, which can terminate at an open distal hole 62 defined by a distally tapered and chamfered distal tip 63 of the catheter body 46. Also, the connector manifold 44 establishes a pathway for fluid communication between the secondary infusion tube 38 and the secondary infusion lumen, which can terminate at an infusion port 64 in a distal segment of the catheter body 46. Additional ports can be provided along the length of the catheter.

The preferred non-limiting catheter 18 has a distally-located heat exchange member for effecting heat exchange with blood as it flows past the catheter when the catheter is positioned in the vasculature of a patient. The heat exchange member can be any of the heat exchange members disclosed in the above-referenced patents. By way of example, the preferred non-limiting catheter shown in FIG. 1 can have proximal and distal thin-walled heat exchange membranes 66, 68 that are arranged along the last fifteen or so centimeters of the catheter body 46 and that are bonded to the outer surface of the catheter body 46, with the infusion port 64 being located between the heat exchange membranes 66, 68. Thus, each preferred non-limiting heat exchange membrane is about six centimeters to seven and one-half centimeters in length, with the heat exchange membranes being longitudinally spaced from each other along the catheter body 46 in the preferred embodiment shown. Essentially, the heat exchange membranes 66, 68 extend along most or all of that portion of the catheter 46 that is intubated within the patient. The heat exchange membranes can be established by a medical balloon material.

The heat exchange membranes 66, 68 can be inflated with heat exchange fluid from the heat exchange fluid source 12 as supplied from the heat exchange fluid supply lumen, and heat exchange fluid from the heat exchange membranes 66, 68 is returned via the heat exchange fluid return lumen to the heat exchange fluid source 12. In their inflated configurations, the heat exchange membranes define a diameter of about ten French, and preferably no more than twelve French. Thus, the heat exchange membranes 66, 68 are relatively long and comparatively thin, to advantageously avoid excessively blocking blood flow through the vena cava while nevertheless effecting patient cooling.

If desired, a temperature sensor 70 such as a thermistor or other suitable device can be attached to the catheter 18 as shown. The sensor 70 can be mounted on the catheter 18 by solvent bonding at a point that is proximal to the membranes 66, 68. Or, the sensor 70 can be disposed in a lumen of the catheter 18, or attached to a wire that is disposed in a lumen of the catheter 18, with the sensor hanging outside the catheter 18. Alternatively, a separate temperature probe can be used, such as the esophageal probe disclosed in U.S. Pat. No. 6,290,717, incorporated herein by reference. As yet another alternative, a rectal probe or tympanic temperature sensor can be used. In any case, the sensor is electrically connected to the heat exchange fluid source 12 for control of the temperature of the heat exchange fluid as described in various of the above-referenced patents.

As envisioned by the present invention, the structure set forth above can be used in many medical applications to cool a patient and/or to maintain temperature in a normothermic or hypothermic patient, for purposes of improving the medical outcomes of patients on whom, e.g., aneurysm surgery is to be performed, preferably while the patient's temperature is below normal body temperature. The structure can then be used to rewarm the patient in a controlled manner by circulating warm heat exchange fluid through the structure, or by otherwise regulating natural body rewarming by circulating heat exchange fluid that is maintained at an appropriate cool (relative to normal body temperature) or warm (relative to normal body temperature) temperature through the structure.

As another example, head trauma and/or stroke can be treated by and after lowering and maintaining the patient's temperature below normal body temperature. Or, cardiac arrest can be treated while the patient's temperature is below normal body temperature. Yet again, minimally invasive heart surgery can be performed on the patient while the patient's temperature is below normal body temperature. And, myocardial infarction in the patient can be treated while the patient's temperature is below normal body temperature. Also, the present invention understands that for certain patients, e.g., stroke victims, it is important to maintain the temperature of a patient at or below normal body temperature, when the patient runs or attempts to run a fever. For severe ischemic stroke victims, the malady can be treated by maintaining the patients body temperature at a hypothermic level.

FIG. 2 shows details of the heat exchange fluid supply to the balloons 66, 68. As shown, the catheter body 46 is formed with plural relatively small inlet holes 80 inside each balloon (only the balloon 68 shown in FIG. 2 for clarity). The inlet holes form passageways into the balloon 68 from the heat exchange fluid supply lumen of the catheter body 46. A single relatively large outlet hole 82 is also formed in the catheter body 46 for returning fluid. More than one outlet hole can be provided if desired.

With this structure, heat exchange fluid is sprayed into the balloon 68 to more evenly cover the inner surface 84 of the balloon 68 than would otherwise be possible were only a single inlet hole to be used to deliver fluid. This in turn increases the effective heat exchange surface of the balloon 68, improving heat exchange with the patient.

While the particular INTRAVASCULAR HEAT EXCHANGE CATHETER WITH MULTIPLE COOLANT INLET HOLES TO BALLOON as herein shown and described in detail is fully capable of attaining the above-described objects of the invention, it is to be understood that it is the presently preferred embodiment of the present invention and is thus representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more”. All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. §112, sixth paragraph, unless the element is expressly recited using the phrase “means for”.

1-4. (canceled)

5. A system for treating a patient, comprising:
an intravascular heat exchange catheter configured for effecting heat exchange with blood as it flows past the catheter when the catheter is positioned in the vasculature of a patient and heat exchange fluid is circulated through the catheter, the catheter including:
a catheter body;
at least one enclosure-forming structure on the body;
plural heat exchange fluid inlet holes in the catheter body and forming respective passageways for heat exchange fluid to flow from an inlet lumen into the enclosure-forming structure;
at least one outlet hole in the body and forming a fluid passageway from the enclosure-forming structure into the catheter, the outlet hole leading to an outlet lumen separate from the inlet lumen; and
a heat exchanger supplying heat exchange fluid to the catheter and receiving heat exchange fluid from the catheter in a closed circuit wherein the heat exchange fluid includes at least one substance having a freezing point below zero degrees Celsius (0° C.) and water.

6. The system of claim 5, wherein the substance is ethanol.

7. The system of claim 5, wherein the enclosure-forming structure is at least one balloon.

8-12. (canceled)

13. A system for effecting heat exchange with a patient, comprising:
a heat exchange catheter configured for placement in a blood vessel of the patient without blocking the vessel to induce hypothermia in the patient when heat exchange fluid is circulated through the catheter, the catheter including:
a catheter body;
at least one enclosure-forming structure on the body;
plural heat exchange fluid inlet holes in the catheter body and forming respective passageways for heat exchange fluid into the enclosure-forming structure; and
at least one outlet hole in the body and forming a fluid passageway from the enclosure-forming structure into the catheter, wherein the inlet holes are smaller than the outlet hole.

14. The system of claim 13, further comprisung a heat exchanger supplying heat exchange fluid to the catheter and receiving heat exchange fluid from the catheter in a closed circuit, the heat exchange fluid including at least one substance having a freezing point below zero degrees Celsius (0° C.) and water.

15-17. (canceled)
